(19) United States
(12) Patent Application Publication
(10) Pub. No.: US 2009/0182299 A1
(43) Pub. Date: Jul. 16, 2009

(54) Absorbent Garment

(75) Inventors: Kristen Brinkdopke, Appleton, WI (US); Marisa Martinez, (US); David J. Krysiak, Menasha, WI (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 12/251,938

(22) Filed: Oct. 15, 2008

Related U.S. Application Data
(60) Provisional application No. 60/984,961, filed on Nov. 2, 2007.

(51) Int. Cl. A61F 13/64; A61F 13/62

(52) U.S. Cl. 604/391; 604/392

(57) ABSTRACT

An absorbent garment is disclosed that may generally fit below a substantial portion of the abdomen of the wearer. The absorbent garment, in one embodiment, is designed to have a relatively short crotch length. In this regard, the front edge of the garment can reside below the abdomen along the inguinal ligament line of the wearer. The garment can have a longer crotch length in the back so as to cover the psis and sacral triangle of the wearer. In accordance with the present disclosure, two belt members extend from the back portion of the garment to the front portion and hold the garment in place.

### RELATED APPLICATIONS

The present application is based upon and claims priority to U.S. Provisional Patent Application No. 60/984,961, filed on Nov. 2, 2007.

### BACKGROUND

Absorbent garments such as adult incontinence products, diapers, training pants, swim pants, fitted briefs, feminine hygiene products, and the like conventionally include a liquid permeable body-facing liner, a liquid impermeable outer cover, and an absorbent core. The absorbent core is typically located in between the outer cover and the liner for taking in and retaining liquids, such as urine, exuded by the wearer.

In many embodiments, the absorbent garments may contain various elastic materials to permit some expansion of the article when necessary and/or to provide a better fit on the wearer. For example, some absorbent garments have been made in the past containing waist elastic members that allow the waist of the article to expand and contract. Absorbent garments have also been made with elastic side panels that allow the garments to expand around the hip and leg of a wearer.

In the past, many of the absorbent garments were designed to fit above the abdomen of the wearer in order to maintain product placement. Certain consumers, especially adult consumers, however, may find such products uncomfortable and/or not aesthetically pleasing when worn. Instead, these consumers may prefer an incontinence product that looks and feels like traditional underwear that fits below the extended abdomen.

As such, a need exists for an absorbent garment that has a relatively shorter crotch depth and thus fits below the abdomen of the wearer while still providing the necessary liquid absorbent capacity.

### SUMMARY

In general, the present disclosure is directed to an absorbent garment that fits generally below the abdomen of the wearer. For example, in one embodiment, the absorbent garment may have a relatively short crotch depth and may include one or more elastic belts that maintain product placement while providing the product with a slim and concise design. The absorbent garments can be made from cloth-like materials to further enhance the aesthetic appeal of the garment and/or to improve comfort.

In one embodiment, for instance, the absorbent garment includes a chassis containing an absorbent core. The chassis can include a front portion, a back portion, and a crotch portion positioned in between the front portion and the back portion. The chassis may further include a pair of opposing leg elastics positioned adjacent to opposite sides of the crotch portion.

In accordance with the present disclosure, the absorbent garment can further include a first elastic belt member and a second elastic belt member. The first elastic belt member, for instance, may extend from the back portion of the chassis to the front portion and form a first side of the garment. The second belt member, on the other hand, can also extend from the back portion of the chassis to the front portion and form a second side of the garment. In this manner, the belt members form a waist opening with the front and back portions of the chassis. Further, the belt portions assist in defining first and second leg openings in conjunction with the crotch portion.

Incorporation of the belt members into the absorbent article in conjunction with having the narrowest part of the crotch portion shifted forward allows for the construction of the garment to fall generally below the abdomen of the wearer when donned. For instance, in one embodiment, the absorbent garment has a shape such that a top edge of the front portion of the chassis generally lies across or near to the inguinal ligament line of a wearer, while the top edge of the back portion sits above the psis and the sacral triangle of a wearer. Thus, the garment can be configured to have a relatively low fit in the front, while ascending in height in the back so as to properly cover one's buttocks.

The belt members can be made from any suitable material. The belt members, for instance, can be made from an extensible material or an elastic material. For instance, in one embodiment, the first and second belt members can be made from a stretch bonded laminate or a neck bonded laminate. As used herein, a stretch bonded laminate refers to a composite material having at least two layers in which one layer is a nonelastic gatherable layer and the other layer is an elastic layer. The layers are joined together when the elastic layer is in an extended condition so that upon relaxing the layers, the gatherable layer is gathered. For example, one elastic member can be bonded to another member while the elastic member is extended at least about 25 percent of its relaxed length. Such a multilayer composite elastic material may be stretched until the nonelastic layer is fully extended. One type of stretch-bonded laminate is disclosed, for example, in U.S. Pat. No. 4,720,415 to Vander Wielen et al., which is incorporated herein by reference. Other composite elastic materials are described and disclosed in U.S. Pat. No. 4,789,699 to Kieffer et al., U.S. Pat. No. 4,781,966 to Taylor, U.S. Pat. No. 4,657,802 to Morman, and U.S. Pat. No. 4,655,760 to Morman et al., all of which are incorporated herein by reference thereto.

As used herein, a neck bonded laminate refers to an elastic member being bonded to a non-elastic member while the non-elastic member is extended in the machine direction creating a necked material. “Neck-bonded laminate” refers to a composite material having at least two layers in which one layer is a necked, non-elastic layer and the other layer is an elastic layer thereby creating a material that is elastic in the cross direction. Examples of neck-bonded laminates are such as those described in U.S. Pat. Nos. 5,226,992, 4,981,747, 4,965,122, and 5,336,545, all to Morman, all of which are incorporated herein by reference thereto.

For example, in one embodiment, the first and second belt members may comprise a stretch bonded laminate comprised of elastic strands positioned between two gathered layers. The elastic strands, for instance, can be made from lycra, while the gathered layers may comprise spunbond webs.

In one particular embodiment, the first and second belt members comprise separate components. Each belt member, for instance, can include a first end and a second and opposite end. The second end of each belt member can be fixedly attached to the back portion of the chassis. For example, the second end of each belt member may be attached in between the outer cover and the liner of the chassis at the back portion.

The first end of each belt member, on the other hand, may include a fastener that allows the first end to be releasably attached to the front portion of the chassis. For example, in one embodiment, the fastener may comprise a hook material configured to attach to the front portion. In one embodiment, for instance, a corresponding loop material may be present on the front portion for attaching the belt members. In this manner, the belt members are adjustable for improving the fit of the garment.

Alternatively, the first end of each belt member can be fixedly attached to the front portion of the chassis, while the second end of each belt member can be releasably attached to the back portion of the chassis.

In an alternative embodiment, the first and second belt members may be integral with each other and may define part of an elastic belt. The elastic belt, for instance, may extend around the back portion and may include two free ends that are attachable to the front portion of the chassis.

The dimensions of the belt member may vary depending upon the particular application, the size of the garment, and various other factors. In general, the belt members have a relatively narrow width. For instance, the first and second belt members can have a width of less than about 100 mm, such as less than about 80 mm. For instance, the belt members may have a width of from about 5 mm to about 80 mm, such as from about 25 mm to about 75 mm.

As described above, in one embodiment, the front edge of the front portion of the chassis may lie below the abdomen of the wearer while the back portion is positioned higher on the wearer in relation to the front edge. For example, in one embodiment, the crotch portion can define a midline. As used herein, the midline of the crotch portion refers to the lowest part of the garment when the garment is being worn. In accordance with the present disclosure, the garment can have dimensions such that the distance from the midline of the crotch portion to the front edge of the front portion can be at least about 5%, such as at least 10% less than the distance from the midline to a back edge of the back portion. For example, the distance from the midline of the crotch portion to the front edge of the front portion can be from about 10% to about 30%, such as from about 12% to about 20% less than the distance from the midline to a back edge of the back portion.

The absorbent garment can comprise any suitable absorbent article. For instance, in one embodiment, the absorbent garment comprises an adult incontinence product. The teachings of the present disclosure, however, may be equally applicable to the construction of diapers, training pants and other similar products.

Other features and aspects of the present disclosure are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a rear view of one embodiment of an absorbent garment made in accordance with the present disclosure;

FIG. 2 is a rear view of another embodiment of an absorbent garment made in accordance with the present disclosure;

FIG. 3 is a front view of the absorbent garment illustrated in FIG. 2;

FIG. 4 is a side view of the absorbent garment illustrated in FIG. 2; and

FIG. 5 is a plan view of the absorbent garment illustrated in FIG. 1.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present invention.

In general, the present disclosure is directed to a low rising absorbent garment that may have an underwear-like shape and look. For instance, in one embodiment, the absorbent garment has a relatively short crotch depth causing the front edge of the garment to rest generally below the abdomen of the wearer. In the back, however, the garment rests higher on the wearer than in the front. In order to secure the garment to the wearer, the garment can include one or more belt members that extend from the rear of the garment to the front of the garment and form the sides of the garment leaving most of the leg and thigh exposed.

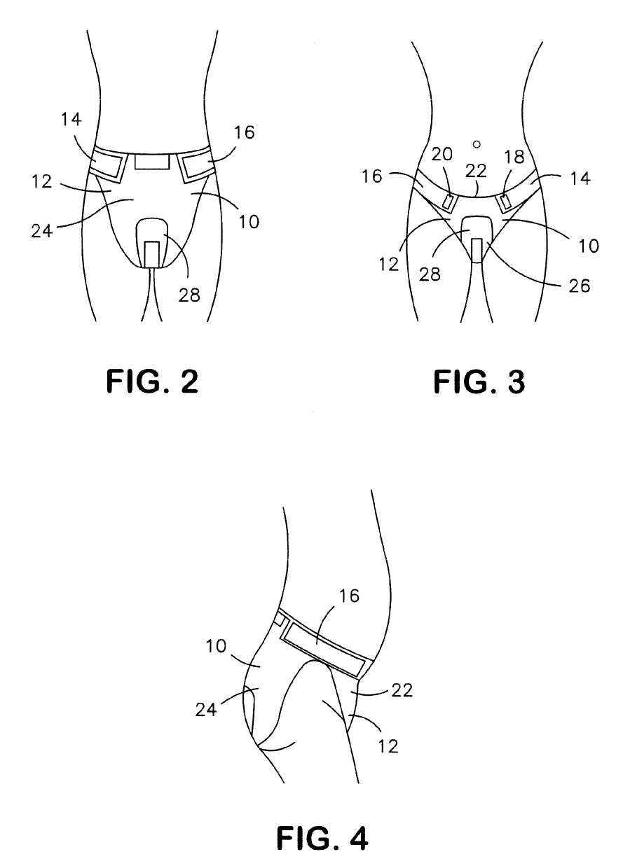

For example, one embodiment of an absorbent garment 10 made in accordance with the present disclosure is shown in FIGS. 2, 3 and 4. In the figures, the absorbent garment is shown on a wearer. A back view of the garment, for instance, is shown in FIG. 2, while a front view of the garment is shown in FIG. 3. FIG. 4, on the other hand, shows a side view of the absorbent garment 10.

The absorbent garment 10 includes a chassis 12 that is comprised of a front portion 22, a back portion 24, and a crotch portion 26. Positioned within the crotch portion 26 and extending from the front portion 22 to the back portion 24 is an absorbent core 28. In addition, the absorbent garment 12 further includes a first belt member 14 and a second belt member 16. The belt members 14 and 16 extend from the rear portion 24 to the front portion 22.

As shown particularly in FIGS. 3 and 4, the absorbent garment 10 has a relatively short crotch depth in the front allowing the front edge of the garment to fit below the abdomen of the wearer. In order to provide sufficient coverage in the back, as shown in FIGS. 2 and 4, the absorbent garment 10 generally has a longer crotch depth in the back.

Each of the belt members 14 and 16 allows the product to have a more underwear-like shape and look while also functionally keeping the product in place when worn. As also shown in the figures, the chassis 12 has leg cutouts and an absorbent core that are shifted forward with respect to the silhouette of the chassis. This design allows the front edge of the absorbent garment 10 to generally fit across the line of the inguinal ligament of the wearer, with the belt members 14 and 16 continuing above the line of the thigh across the iliac line of the wearer. Ultimately, the belt members 14 and 16 extend to the back portion 24 of the chassis 12 which fits higher on the body thereby covering the psis and sacral triangle of the wearer.

In one embodiment, the belt members 14 and 16 can be adjustable so as to allow the wearer to adjust the amount of tension around the waist opening of the garment. For instance, as shown in FIG. 3, the first belt member 14 can include a first fastener 18, while the second belt member 16 can include a second fastener 20. The fasteners 18 and 20 can be configured to releasably attach the belt members 14 and 16 to the front portion 22 of the chassis 12. For instance, as will be described in greater detail below, the fasteners 18 and 20 can comprise a hook material that is configured to engage a suitable loop material located on the chassis. By making the belt members 14 and 16 adjustable, a user can ensure that the waist opening fits appropriately across the iliac line of the wearer by increasing or decreasing the tension in the belt members at their distal end.

As shown, for instance, in FIG. 4, the belt members 14 and 16 not only connect the front portion of the chassis to the back portion, but also form the sides of the garment. The belt members 14 and 16, for instance, define a waist opening with the chassis 12. In addition, the belt members 14 and 16 also define the leg opening of the absorbent garment 10 in conjunction with the crotch portion 26. As illustrated in FIG. 4, the garment in one embodiment can form relatively large leg openings that leave most of the leg uncovered. The leg openings in the back, however, can be configured to extend across the gluts to the gluteal fold in order to provide an amount of coverage necessary to cover the rear of the wearer while providing sufficient comfort. As shown, the leg openings can also stay to the lateral side of the most extended portion of the gluts to prevent the chassis from shifting into the gluteal cleft.

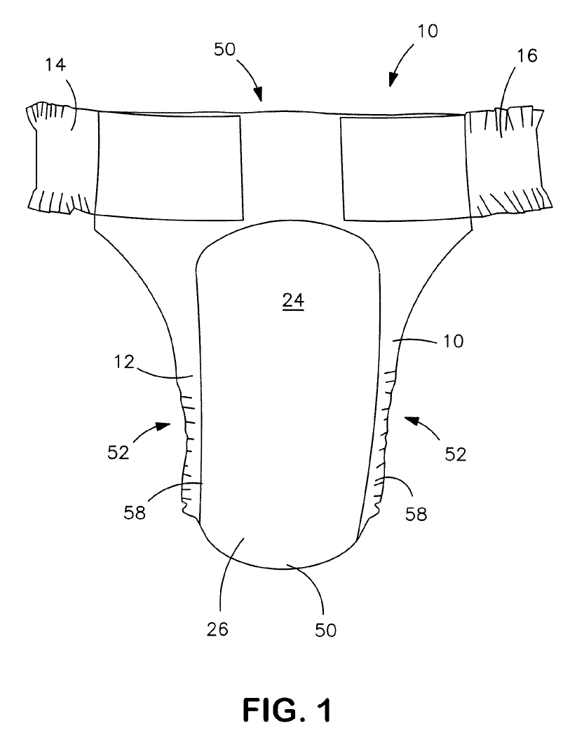

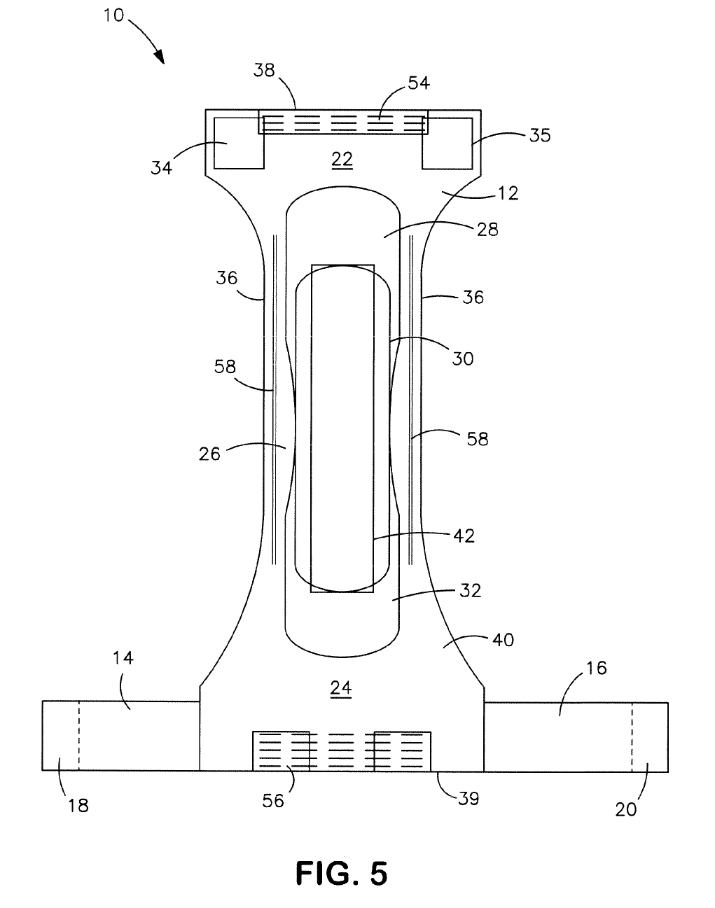

Referring to FIGS. 1 and 5, one embodiment of an absorbent garment 10 generally made in accordance with the present disclosure will now be described in greater detail. In FIG. 1, the back of the absorbent garment 10 is shown. In FIG. 5, on the other hand, the absorbent garment 10 is shown in an opened and unfolded state. Specifically, FIG. 5 is a plan view illustrating the exterior side of the garment. As shown in FIG. 5, the absorbent garment 10 generally extends in a longitudinal direction from the back portion 24 to the front portion 22. Opposite to the longitudinal direction is a lateral direction. As shown, the absorbent garment includes a pair of laterally opposite side edges 36, a front waist edge 38, and a back waist edge 39.

The chassis 12 of the absorbent garment 10 can be formed from an outer cover 40 and a bodyside liner that may be joined to the outer cover 40 in a superimposed relation. The outer cover and the liner, for instance, may be joined together by adhesives, ultrasonic bonds, thermal bonds, or other conventional techniques. For instance, the liner may be suitably joined to the outer cover 40 along the perimeter of the chassis 12. The liner can be positioned relative to the other components of the garment so as to be disposed toward the wearer's skin when the garment is being worn.

As shown in FIG. 5, the chassis 12 may further include an absorbent core 28 disposed between the outer cover 40 and the bodyside liner for absorbing liquid body exudates exuded by the wearer. In one embodiment, the absorbent core 28 may have a zoned absorbent core. For instance, as shown in FIG. 5, the absorbent core 28 may include an inner zone 30 surrounded by an outer zone 32. The inner zone 30, for instance, may have greater absorbent capacity than the outer zone 32. The inner zone 30, for instance, may be configured to quickly absorb liquids for movement to the outer zone 32.

As shown in FIG. 1, the front portion and the back portion 24 of the chassis 12 are connected together by the belt members 14 and 16 to define a three-dimensional configuration having a waist opening 50 and a pair of leg openings 52. As shown in FIGS. 1 and 5, the leg openings 52 can be surrounded by leg elastic members 58. In addition, as shown in FIG. 5, to further enhance containment and/or absorption of body exudates and/or to improve comfort, the absorbent garment 10 may further include a front waist elastic member 54 and/or a rear waist elastic member 56. The waist elastic members 54 and 56 can be operatively joined to the outer cover 40 and/or the bodyside liner and can extend over part or all of the waist edges 38 and 39. The leg elastic members 58 can be operatively joined to the outer cover 40 and/or the bodyside liner and positioned in the crotch portion 26 of the absorbent garment 10.

The waist elastic members 54 and 56, and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA and available from Invista, Wilmington, Del., U.S.A.

In addition to the leg elastics 58, in one embodiment, the absorbent garment may include containment flaps that define a partially unattached edge and extend over the crotch portion 26 of the chassis. The containment flaps, for instance, may be connected to the bodyside liner and can define a partially unattached edge which assumes an upright configuration in at least the crotch portion of the garment to form a seal against the wearer's body. Suitable constructions and arrangements for containment flaps are generally well known to those skilled in the art and are described, for instance, in U.S. Pat. No. 4,704,116, which is incorporated herein by reference.

Referring to FIG. 5, in one embodiment, the absorbent garment 10 may further include a surge management layer 42 which may be located adjacent to the liner and/or the absorbent core 28. A surge management layer helps to decelerate and diffuse surges or gushes of liquid that may be rapidly introduced into the absorbent core. Desirably, the surge management layer can rapidly accept and temporarily hold the liquid prior to releasing the liquid into the storage or retention portions of the absorbent core. In one embodiment, for instance, the surge management layer 42 may be formed from a nonwoven web containing bicomponent fibers. The surge management layer, for instance, can have a basis weight of from about 40 gsm to about 100 gsm. Other examples of surge management layers are described in U.S. Pat. No. 5,486,166, U.S. Pat. No. 5,490,846 and U.S. Pat. No. 5,820,973, which are all incorporated herein by reference.

As shown in FIG. 5, the front portion 22 and the back portion 26 are generally wider than the crotch portion 26. The side edges 36 of the absorbent garment 10 may be suitably curved and/or angled relative to the lateral direction to provide a better fit around the wearer's legs. The waist edges 38 and 39 are generally parallel to the transverse axis of the garment.

The belt members 14 and 16 used to attach the front portion 22 to the back portion 24 are generally made from any suitable elastic material capable of stretching at least in a direction generally parallel to the lateral direction of the garment. Suitable elastic materials that may be used to form the belt members are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference to the extent that they are consistent (i.e., not in conflict) herewith.

In one particular embodiment, for instance, the belt members 14 and 16 (and also the waist elastic members 54 and 56) can be formed from a stretch bonded laminate containing an elastic strand layer positioned in between two gathered layers that allow the strand layer to stretch.

The strand layer can generally contain a plurality of elastic strands positioned in parallel. The number of strands may vary as desired, such as from 3 to about 20, in some embodiments from about 7 to about 18, and in some embodiments, from about 8 to 15 strands per cross-directional inch. The strands may have a circular cross-section, but may alternatively have other cross-sectional geometries such as elliptical, rectangular as in ribbon-like strands, triangular, multi-lobal, etc. The diameter of the strands (the widest cross-sectional dimension) may vary as desired, such as within a range of from 0.1 to about 4 millimeters, in some embodiments from about 0.2 to about 2.5 millimeters, and in some embodiments, from 0.5 to about 2 millimeters. Further, the elastic strands may generally be arranged in any direction or pattern. For example, in one embodiment, the strands are arranged in a direction that is substantially parallel to the lateral direction of the garment and are desirably spaced apart from each other at similar intervals.

If desired, the elastic strands may be substantially continuous in length so that they are in the form of filaments. Such filaments may be produced using any of a variety of known techniques, such as by extruding an elastomeric polymeric composition from a die having a series of extrusion capillaries arranged in a row. As is well known in the art, meltblown dies may be suitable for forming the filaments, except that the high velocity gas streams used in fiber attenuation are generally not employed. Rather, the molten polymer extrudate is pumped from the die capillaries and allowed to extend away from the die under the impetus of gravity. Besides extruded filaments, other elastic filaments may also be employed in the present invention, such as the spandex-type materials available under the designation “LYCRA®” from Invista North America of Wilmington, Del.

Regardless of the particular configuration of the strands, the elastic strand layer of the present invention employs a combination of a thermoplastic elastomer and a semi-crystalline polyolefin. Any of a variety of thermoplastic elastomers may generally be employed, such as elastomeric polyesters, elastomeric polyurethanes, elastomeric polyamides, elastomeric copolymers, and so forth, may be employed in some embodiments of the present invention. For example, the thermoplastic elastomer may be a substantially amorphous block copolymer having at least two blocks of a monoalkenyl arene polymer separated by at least one block of a saturated conjugated diene polymer. The monoalkenyl arene blocks may include styrene and its analogues and homologues, such as o-methyl styrene; p-methyl styrene; p-tert-butyl styrene; 1,3 dimethyl styrene p-methyl styrene; etc., as well as other monoalkenyl polycyclic aromatic compounds, such as vinyl naphthalene; vinyl anthrycene; and so forth. Preferred monoalkenyl arenes are styrene and p-methyl styrene. The conjugated diene blocks may include homopolymers of conjugated diene monomers, copolymers of two or more conjugated dienes, and copolymers of one or more of the dienes with another monomer in which the blocks are predominantly conjugated diene units. Preferably, the conjugated dienes contain from 4 to 8 carbon atoms, such as 1,3 butadiene (butadiene); 2-methyl-1,3 butadiene; isoprene; 2,3 dimethyl-1,3 butadiene; 1,3 pentadiene (piperylene); 1,3 hexadiene; and so forth.

The amount of monoalkenyl arene (e.g., polystyrene) blocks may vary, but typically constitute from about 8 wt. % to about 55 wt. %, in some embodiments from about 10 wt. % to about 35 wt. %, and in some embodiments, from about 25 wt. % to about 35 wt. % of the copolymer. Suitable block copolymers may contain monoalkenyl arene endblocks having a number average molecular weight from about 5,000 to about 35,000 and saturated conjugated diene midblocks having a number average molecular weight from about 20,000 to about 170,000. The total number average molecular weight of the block polymer may be from about 30,000 to about 250,000.

Particularly suitable thermoplastic elastomers are available from Kraton Polymers LLC of Houston, Tex. under the trade name KRATON®. KRATON® polymers include styrene-diene block copolymers, such as styrene-butadiene, styrene-isoprene, styrene-butadiene-styrene, and styrene-isoprene-styrene. KRATON® polymers also include styrene-olefin block copolymers formed by selective hydrogenation of styrene-diene block copolymers. Examples of such styrene-olefin block copolymers include styrene-(ethylene-butylene), styrene-(ethylene-propylene), styrene-(ethylene-butylene)-styrene, styrene-(ethylene-propylene)-styrene, styrene-(ethylene-butylene)-styrene-(ethylene-butylene), styrene-(ethylene-propylene)-styrene-(ethylene-propylene), and styrene-ethylene-(ethylene-propylene)-styrene. These block copolymers may have a linear, radial or star-shaped molecular configuration. Specific KRATON® block copolymers include those sold under the brand names G 1652, G 1657, G 1730, MD6673, and MD6937. Various suitable styrenic block copolymers are described in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Other exemplary thermoplastic elastomers that may be used include polyurethane elastomeric materials such as, for example, those available under the trademark ESTANE from Noveon and LYCRA from Invista, polyamide elastomeric materials such as, for example, those available under the trademark PEBAX (polyether amide) from Atofina Chemicals Inc., of Philadelphia, Pa., and polyester elastomeric materials such as, for example, those available under the trade designation HYTREL from E.I. DuPont De Nemours & Company.

The semi-crystalline polyolefin of the elastic strand layer has or is capable of exhibiting a substantially regular structure. That is, semi-crystalline polyolefins may be substantially amorphous in their undeformed state, but form crystalline domains upon stretching. The degree of crystallinity of the olefin polymer may be from about 3% to about 30%, in some embodiments from about 5% to about 25%, and in some embodiments, from about 5% and about 15%. Likewise, the semi-crystalline polyolefin may have a latent heat of fusion (ΔH_{f}), which is another indicator of the degree of crystallinity, of from about 15 to about 75 Joules per gram (“J/g”), in some embodiments from about 20 to about 65 J/g, and in some embodiments, from 25 to about 50 J/g. The semi-crystalline polyolefin may also have a Vicat softening temperature of from about 10° C. to about 100° C., in some embodiments from about 20° C. to about 80° C., and in some embodiments, from about 30° C. to about 60° C. The semi-crystalline polyolefin may have a melting temperature of from about 20° C. to about 120° C., in some embodiments from about 35° C. to about 90° C., and in some embodiments, from about 40° C. to about 80° C. The latent heat of fusion (ΔH_{f}) and melting temperature may be determined using differential scanning calorimetry (“DSC”) in accordance with ASTM D-3417 as is well known to those skilled in the art. The Vicat softening temperature may be determined in accordance with ASTM D-1525.

Exemplary semi-crystalline polyolefins include polyethylene, polypropylene, blends and copolymers thereof. In one particular embodiment, a polyethylene is employed that is a copolymer of ethylene and an α-olefin, such as a C₃-C₂₀ α-olefin or C₃-C₁₂ α-olefin. Suitable α-olefins may be linear or branched (e.g., one or more C₁-C₃ alkyl branches, or an aryl group). Specific examples include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-dimethyl-substituted 1-decene; 1-dodecene; and styrene. Particularly desired α-olefin comonomers are 1-butene, 1-hexene and 1-octene. The ethylene content of such copolymers may be from about 60 mole % to about 99 mole %, in some embodiments from about 80 mole % to about 98.5 mole %, and in some embodiments, from about 87 mole % to about 97.5 mole %. The α-olefin content may likewise range from about 1 mole % to about 40 mole %, in some embodiments from about 1.5 mole % to about 15 mole %, and in some embodiments, from about 2.5 mole % to about 13 mole %.

The density of the polyethylene may vary depending on the type of polymer employed, but generally ranges from 0.85 to 0.96 grams per cubic centimeter (“g/cm³”). Polyethylene “plastomers”, for instance, may have a density in the range of from 0.85 to 0.91 g/cm³. Likewise, “linear low density polyethylene” (“LLDPE”) may have a density in the range of from 0.91 to 0.940 g/cm³; “low density polyethylene” (“LDPE”) may have a density in the range of from 0.910 to 0.940 g/cm³; and “high density polyethylene” (“HDPE”) may have density in the range of from 0.940 to 0.960 g/cm³. Densities may be measured in accordance with ASTM 1505.

Particularly suitable polyethylene copolymers are those that are “linear” or “substantially linear.” The term “substantially linear” means that, in addition to the short chain branches attributable to comonomer incorporation, the ethylene polymer also contains long chain branches in the polymer backbone. “Long chain branching” refers to a chain length of at least 6 carbons. Each long chain branch may have the same comonomer distribution as the polymer backbone and be as long as the polymer backbone to which it is attached. Preferred substantially linear polymers are substituted with from 0.01 long chain branch per 1000 carbons to 1 long chain branch per 1000 carbons, and in some embodiments, from 0.05 long chain branch per 1000 carbons to 1 long chain branch per 1000 carbons. In contrast to the term “substantially linear”, the term “linear” means that the polymer lacks measurable or demonstrable long chain branches. That is, the polymer is substituted with an average of less than 0.01 long chain branch per 1000 carbons.

The density of a linear ethylene/α-olefin copolymer is a function of both the length and amount of the α-olefin. That is, the greater the length of the α-olefin and the greater the amount of α-olefin present, the lower the density of the copolymer. Although not necessarily required, linear polyethylene “plastomers” are particularly desirable in that the content of α-olefin short chain branching content is such that the ethylene copolymer exhibits both plastic and elastomeric characteristics—i.e., a “plastomer.” Because polymerization with α-olefin comonomers decreases crystallinity and density, the resulting plastomer normally has a density lower than that of polyethylene thermoplastic polymers (e.g., LLDPE), but approaching and/or overlapping that of an elastomer. For example, the density of the polyethylene plastomer may be about 0.91 grams per cubic centimeter (g/cm³) or less, in some embodiments from about 0.85 to about 0.88 g/cm³, and in some embodiments, from about 0.85 g/cm³ to about 0.87 g/cm³. Despite having a density similar to elastomers, plastomers generally exhibit a higher degree of crystallinity, are relatively non-tacky, and may be formed into pellets that are non-adhesive and relatively free flowing.

The distribution of the α-olefin comonomer within a polyethylene plastomer is typically random and uniform among the differing molecular weight fractions forming the ethylene copolymer. This uniformity of comonomer distribution within the plastomer may be expressed as a comonomer distribution breadth index value (“CDBI”) of 60 or more, in some embodiments 80 or more, and in some embodiments, 90 or more. Further, the polyethylene plastomer may be characterized by a DSC melting point curve that exhibits the occurrence of a single melting point peak occurring in the region of 50 to 110° C. (second melt rundown).

Plastomers for use in the present disclosure are ethylene-based copolymer plastomers available under the AFFINITY™ from Dow Chemical Company of Midland, Michiqan. Other suitable polyethylene plastomers are available under the designation ENGAGE™ from Dow Chemical Company of Midland, Mich. and EXACT™ from ExxonMobil Chemical Company of Houston, Tex. Still other suitable ethylene polymers are available from The Dow Chemical Company under the designations DOWLEX™ (LLDPE) and ATTANE™ (ULDPE). Other suitable ethylene polymers are described in U.S. Pat. No. 4,937,299 to Ewen et al.; U.S. Pat. No. 5,218,071 to Tsutsui et al.; U.S. Pat. No. 5,272,236 to Lai, et al.; and U.S. Pat. No. 5,278,272 to Lai, et al., which are incorporated herein in their entirety by reference thereto for all purposes.

Propylene polymers may also be suitable for use as a semi-crystalline polyolefin. In one particular embodiment, the semi-crystalline propylene-based polymer includes a copolymer of propylene and an α-olefin, such as a C₂-C₂₀ α-olefin or C₂-C₁₂ α-olefin, Particularly desired α-olefin comonomers are ethylene, 1-butene, 1-hexene and 1-octene. The propylene content of such copolymers may be from about 60 mole % to about 99.5 wt. %, in some embodiments from about 80 mole % to about 99 mole %, and in some embodiments, from about 85 mole % to about 98 mole %. The α-olefin content may likewise range from about 0.5 mole % to about 40 mole %, in some embodiments from about 1 mole % to about 20 mole %, and in some embodiments; from about 2 mole % to about 15 mole %. The distribution of the α-olefin comonomer is typically random and uniform among the differing molecular weight fractions forming the propylene copolymer. Although the density of the propylene-based polymer employed in the present invention may vary, it is typically about 0.91 grams per cubic centimeter (g/cm³) or less, in some embodiments from about 0.85 to about 0.88 g/cm³, and in some embodiments, from about 0.85 g/cm³ to about 0.87 g/cm³. The melt flow rate of the propylene-based polymer may also be selected within a certain range to optimize the properties of the resulting elastic material. The melt flow rate is the weight of a polymer (in grams) that may be forced through an extrusion rheometer orifice (0.0825-inch diameter) when subjected to a force of 2160 grams in 10 minutes at 230° C. Generally speaking, the melt flow rate is high enough to improve melt processability, but not so high as to adversely interfere with binding properties. Thus, in most embodiments of the present invention, the propylene-based polymer has a melt flow index of from about 0.1 to about 10 grams per 10 minutes, in some embodiments from about 0.2 to about 5 grams per 10 minutes, and in some embodiments, from about 0.5 to about 4 grams per 10 minutes, measured in accordance with ASTM Test Method D1238-E.

Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes.

Any of a variety of known techniques may generally be employed to form the semi-crystalline polyolefins. For instance, olefin polymers may be formed using a free radical or a coordination catalyst (e.g., Ziegler-Natta). Preferably, the olefin polymer is formed from a single-site coordination catalyst, such as a metallocene catalyst. Such a catalyst system produces ethylene copolymers in which the comonomer is randomly distributed within a molecular chain and uniformly distributed across the different molecular weight fractions. Metallocene-catalyzed polyolefins are described, for instance, in U.S. Pat. No. 5,571,619 to McAlpin et al.; U.S. Pat. No. 5,322,728 to Davis et al.; U.S. Pat. No. 5,472,775 to Obiueski et al.; U.S. Pat. No. 5,272,236 to Lai et al.; and U.S. Pat. No. 6,090,325 to Wheat, et al., which are incorporated herein in their entirety by reference thereto for all purposes. Examples of metallocene catalysts include bis(n-butylcyclopentadienyl)titanium dichloride, bis(n-butylcyclopentadienyl)zirconium dichloride, bis(cyclopentadienyl)scandium chloride, bis(indenyl)zirconium dichloride, bis(methylcyclopentadienyl)titanium dichloride, bis(methylcyclopentadienyl)zirconium dichloride, cobaltocene, cyclopentadienyltitanium trichloride, ferrocene, hafnocene dichloride, isopropyl(cyclopentadienyl, -1-flourenyl)zirconium dichloride, molybdocene dichloride, nickelocene, niobocene dichloride, ruthenocene, titanocene dichloride, zirconocene chloride hydride, zirconocene dichloride, and so forth. Polymers made using metallocene catalysts typically have a narrow molecular weight range. For instance, metallocene-catalyzed polymers may have polydispersity numbers (M_{w}/Mₙ) of below 4, controlled short chain branching distribution, and controlled isotacticity.

The melt flow index (Ml) of the semi-crystalline polyolefins may generally vary, but is typically in the range of about 0.1 grams per 10 minutes to about 100 grams per 10 minutes, in some embodiments from about 0.5 grams per 10 minutes to about 30 grams per 10 minutes, and in some embodiments, about 1 to about 10 grams per 10 minutes, determined at 190° C. The melt flow index is the weight of the polymer (in grams) that may be forced through an extrusion rheometer orifice (0.0825-inch diameter) when subjected to a force of 2.16 kilograms in 10 minutes at 190° C., and may be determined in accordance with ASTM Test Method D1238-E.

The relative amounts of the thermoplastic elastomers and semi-crystalline polyolefins are selectively controlled in accordance with the present invention to achieve a balance between the mechanical and thermal properties of the elastic strand layer. For example, the ratio of the amount of the thermoplastic elastomer(s) to the amount of the semi-crystalline polyolefin(s) may range from about 0.5 to about 15, in some embodiments from about 1 to about 10, and in some embodiments, from about 1 to about 5. The thermoplastic elastomer(s) may constitute from about 40 wt. % to about 95 wt. %, in some embodiments from about 45 wt. % to about 90 wt. %, and in some embodiments, from about 50 wt. % to about 75 wt. % of the elastic strand layer. Likewise, the semi-crystalline polyolefin(s) may constitute from about 5 wt. % to about 60 wt. %, in some embodiments from about 10 wt. % to about 55 wt. %, and in some embodiments, from about 15 wt. % to about 50 wt. % of the elastic strand layer. It should of course be understood that other polymers may also be employed in the elastic strand layer. When utilized, however, the other polymers typically constitute about 10 wt. % or less, and in some embodiments, about 5 wt. % or less of the material.

In addition to polymers, the elastic strand layer may also employ other additives as is known in the art. For example, although the elastomeric polymers may possess a certain amount of tack, a tackifying resin may nevertheless be employed in some embodiments to facilitate subsequent bonding of the strand layer to a nonwoven web facing. One suitable class of tackifying resins includes hydrogenated hydrocarbon resins, such as REGALREZ™ hydrocarbon resins available from Eastman Chemical. Other suitable tackifying resins may be described in U.S. Pat. No. 4,787,699. When employed, the tackifying resin may be present in an amount from about 0.001 wt. % to about 35 wt. %, in some embodiments, from about 0.005 wt. % to about 30 wt. %, and in some embodiments, from 0.01 wt. % to about 25 wt. % of the elastic strand layer.

The elastic strand layer may also contain other additives as is known in the art, such as melt stabilizers, processing stabilizers, heat stabilizers, light stabilizers, antioxidants, heat aging stabilizers, whitening agents, antiblocking agents, bonding agents, viscosity modifiers, etc. Viscosity modifiers may also be employed, such as polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of fibers and films. Some suitable hindered phenols include those available from Ciba Specialty Chemicals of under the trade name “Irganox®”, such as Irganox® 1076, 1010, or E 201. Moreover, bonding agents may also be added to facilitate bonding to additional materials (e.g., nonwoven web). When employed, additives (e.g., antioxidant, stabilizer, etc.) may each be present in an amount from about 0.001 wt. % to about 40 wt. %, in some embodiments, from about 0.005 wt. % to about 35 wt. %, and in some embodiments, from 0.01 wt. % to about 25 wt. % of the elastic strand layer.

As described above, the strand layer is laminated to at least one gathered layer. For instance, in one embodiment, the strand layer may be laminated in between a pair of opposing gathered layers. The gathered layers may comprise any suitable nonwoven material. For instance, in one embodiment, the gathered layers may comprise spunbond webs. The gathered layers, for instance, can have a basis weight of from about 5 gsm to about 120 gsm and can comprise a single layer or can comprise multiple layers.

As shown in the figures, in order for the absorbent garment 10 to have an underwear-like look and to provide proper fit below the abdomen of the wearer, the belt members 14 and 16 generally have a relatively narrow width. The belt members 14 and 16, for instance, can have a width of less than about 100 mm, such as less than about 80 mm. For instance, the width of the belt members can be from about 5 mm to about 100 mm, such as from about 25 mm to about 75 mm.

In one particular embodiment, for instance, the belt members are comprised of ten 940 dtex spandex strands positioned between two spunbond layers having a basis weight of about 0.5 osy. In this embodiment, the belt members can have a width of about 70 mm. The length of the belt members can vary depending upon the size of the garment and can, in one embodiment, be between about 290 mm to about 450 mm stretch to stop.

To maintain the look of a continuous waistband, the belt members 14 and 16 can be permanently attached to the back portion 24 of the chassis 12. More particularly, the belt members can be attached directly to the interior of the chassis for a more streamlined look. For example, in one embodiment, the end of each belt member may be deadened by slitting or cutting the elastic filaments over a desired area. The end of the belt members can then be attached in between the outer cover and the liner of the garment using a combination of an adhesive and pressure bond as shown in FIG. 1.

The opposite end of the belt members 14 and 16 may be permanently bonded to the front portion 22 of the chassis 12. Alternatively, as shown in FIG. 5, the first belt member 14 may include a fastener 18, while the second belt member 16 may include a fastener 20 that releasably engages the front portion 22.

The fasteners 18 and 20 may comprise any suitable fasteners, such as adhesive fasteners, cohesive fasteners, mechanical fasteners, or the like. Suitable mechanical fastening elements can be provided by interlocking geometric shaped materials, such as hooks, loops, bulbs, mushrooms, arrowheads, balls on stems, male and female mating components, buckles, snaps, or the like.

In the embodiment illustrated, for instance, the fasteners 18 and 20 may comprise hook fasteners. The hook fasteners may be configured to attach directly to the outer cover 40. Alternatively, the front portion 22 of the chassis 12 may include a complementary loop material, such as 34 and 35 as shown in FIG. 5.

Using a fastening system, such as a hook and loop fastener, allows for the belt members 14 and 16 to be adjusted when the garment 10 is being donned by the wearer.

In one particular embodiment, a hook material may be attached to the belt members. Prior to attaching the hook material, the elastics within the belt members may be deadened by cutting or chopping the elastic strands. The hook material can have any suitable dimension, such as an area of 30 mm×60 mm. The loop material 34 and 35 attached to the front portion of the chassis, on the other hand, may comprise, in one embodiment, a point unbonded material having a length and width of about 50 mm.

In the embodiment illustrated in FIGS. 1 and 5, the belt members 14 and 16 comprise separate components attached to the back portion 24 of the absorbent garment. It should be understood, however, that in an alternative embodiment, the belt members 14 and 16 may be integral with each other and may be part of a continuous belt that extends around the entire back portion 24. In this embodiment, for instance, a rear waist elastic member 56 may be eliminated. When using a continuous belt around the back portion, however, it may be desirable to deaden the elastics within the belt in certain areas to improve comfort. For instance, the elastic within the belt may be deadened along the lateral edges of the back portion 24.

When using a continuous belt, the belt may include a fastening system as shown in FIG. 5 for attachment to the front portion 22. Alternatively, the belt may comprise an endless belt that also surrounds the front portion 22. In still an alternative embodiment, the belt may include free ends that are permanently attached to the front portion.

As shown particularly in FIGS. 2, 3 and 4, when donned, the front edge 38 of the absorbent garment generally lies along the inguinal ligament line of the wearer while the back portion rises higher along the body covering the psis and the sacral triangle of the wearer. Thus, the front of the garment generally has a shorter crotch length than the back of the garment. In this regard, the garment defines a midline 50 as shown in FIG. 1 that generally lies within the crotch portion 26. The midline 50 is defined as the lowest part of the garment when being worn (and thus does not necessarily define the middle of the crotch portion). In accordance with the present disclosure, the length of the garment from the midline 50 to the front edge 38 of the chassis is generally at least about 5% less than the distance from the midline 50 to the back edge 39 of the chassis. For instance, the distance from the midline to the front edge of the chassis can be at least about 10% less, such as at least about 15% less, such as even about 20% less than the distance from the midline to the back edge of the chassis.

As described above, the absorbent garment 10 includes an outer cover, a bodyside liner, and an absorbent core. These elements of the absorbent garment may be made from any suitable materials.

The outer cover may be made from a material that is substantially liquid and permeable, and can be elastic, stretchable or nonstretchable. The outer cover can be a single layer of liquid and permeable material, or may include a multi-layered laminate structure in which at least one of the layers is liquid and permeable. For instance, the outer cover can include a liquid permeable outer layer and a liquid and permeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent core is positioned in between the outer cover and a liquid permeable bodyside liner. The bodyside liner is suitably compliant, soft feeling, and non-irritating to the wearer's skin. The bodyside liner can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner. For example, the bodyside liner can be made from a meltblown or spunbonded web of polyolefin fibers. The bodyside liner can also be a bonded-carded web composed of natural and/or synthetic fibers.

The bodyside liner may be constructed to be extensible but not elastic. In other embodiments, however, the liner may be configured to be elastic in the longitudinal direction, in the transverse direction, or in both directions.

A suitable liquid permeable bodyside liner is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent core, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent core, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference for all purposes.

It is also contemplated that elastomeric absorbent web structures may be used. For example, an elastomeric coform absorbent core may be used to form the absorbent core according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments with the matrix including a plurality of absorbent fibers and a super-absorbent material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

Absorbent garments made in accordance with the present disclosure can be used in numerous applications. For instance, the absorbent garments can be used as a diaper, a training pant, a feminine hygiene product, and the like. In one particular embodiment, for instance, the absorbent garment may comprise an adult incontinence product.

When designed for an adult, for instance, the chassis can have a length of from about 575 mm to about 850 mm, can have a width at the center or crotch portion of from about 100 mm to about 180 mm, and can have a width at the ends of from about 250 mm to about 300 mm. The absorbent core, on the other hand, can have a length of from about 400 mm to about 500 mm, can have a width at the center of from about 70 mm to about 110 mm, and can have a width at the ends of from about 90 mm to about 130 mm.

As described above, in one embodiment, the belt members 14 and 16 comprise elastic strands sandwiched between two gathered layers. In one embodiment, the belt members may be configured such that all or a portion of the strands are visible through the gathered layers. The strands can be visible by using strands having a different color than the gathered layers or using gathered layers that are somewhat translucent. The present inventors have discovered that by using a stretch bonded laminate as described above, the belt members have an appearance of a woven, cloth-like material further giving the product an underwear-like look.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An absorbent garment comprising:
a chassis containing an absorbent core and including a front portion, a back portion, and a crotch portion positioned in between the front portion and the back portion;
a first elastic belt member and a second elastic belt member, the first belt member extending from the back portion to the front portion of the chassis thereby forming a first side of the garment, the second belt member also extending from the back portion to the front portion of the chassis thereby forming a second side of the garment, the first and second belt members forming a waist opening with the front and back portions, the first and second belt members forming first and second leg openings respectively with the crotch portion; and
wherein the garment has a shape such that a top edge of the front portion generally lies across or near an inguinal ligament line of a wearer and a top edge of the back portion fits above a psis and sacral triangle of a wearer.

2. An absorbent garment as defined in claim 1, wherein the first and second elastic belt members have a width of less than about 100 mm.

3. An absorbent garment as defined in claim 1, wherein the first and second elastic belt members have a width of less than about 80 mm.

4. An absorbent garment as defined in claim 1, wherein the first and second elastic belt members are made from a stretch bonded laminate.

5. An absorbent garment as defined in claim 4, wherein the stretch bonded laminate comprises elastic strands positioned in between two gathered layers.

6. An absorbent garment as defined in claim 5, wherein the elastic strands comprise lycra and the two gathered layers comprise spunbond webs.

7. An absorbent garment as defined in claim 1, wherein the first and second belt members comprise separate components, each belt member including a first end and a second and opposite end, the second end of each belt member being fixedly attached to the back portion of the chassis.

8. An absorbent garment as defined in claim 7, wherein the first end of each belt member includes a fastener that releasably attaches to the front portion of the chassis.

9. An absorbent garment as defined in claim 1, wherein the first and second belt members are integral comprising a continuous elastic belt that extends around the back portion of the chassis.

10. An absorbent garment as defined in claim 8, wherein the fastener comprises a hook material.

11. An absorbent garment as defined in claim 1, wherein the chassis includes a front edge and a back edge and the crotch portion defines a midline and wherein the distance from the midline to the front edge is at least about 5% less than the distance from the midline to the back edge.

12. An absorbent garment as defined in claim 1, wherein the chassis includes a front edge and a back edge and the crotch portion defines a midline and wherein the distance from the midline to the front edge is at least about 10% less than the distance from the midline to the back edge.

13. An absorbent garment as defined in claim 1, wherein the garment comprises an adult incontinence product.

14. An absorbent garment as defined in claim 7, further comprising a back elastic member located on the back portion adjacent to the waist opening.

15. An absorbent garment as defined in claim 7, further comprising a front elastic member positioned on the front portion adjacent to the waist opening.

16. An absorbent garment as defined in claim 5, wherein the elastic strands contained in the belt members are parallel with respect to each other.

17. An absorbent garment as defined in claim 1, wherein the chassis comprises a liner and an outer cover, the absorbent core being positioned in between the liner and the outer cover.

18. An absorbent garment comprising:
a chassis containing an absorbent core and including a front portion, a back portion, and a crotch portion positioned in between the front portion and the back portion, the chassis further including a pair of opposing leg elastics positioned adjacent to opposite sides of the crotch portion; and
a first elastic belt member and a second elastic belt member, the first belt member extending from the back portion to the front portion of the chassis thereby forming a first side of the garment, the second belt member also extending from the back portion to the front portion of the chassis thereby forming a second side of the garment, the first and second belt members forming a waist opening with the front and back portions, the first and second belt members forming first and second leg openings respectively with the crotch portion, the first and second elastic belt members having a width of less than about 100 mm.

19. An absorbent garment as defined in claim 18, wherein the first and second belt members have a width less than about 80 mm.

20. An absorbent garment as defined in claim 18, wherein the first and second elastic belt members are made from a stretch bonded laminate.

21. An absorbent garment as defined in claim 20, wherein the stretch bonded laminate comprises elastic strands positioned in between two gathered layers.

22. An absorbent garment as defined in claim 18, wherein the first and second belt members comprise separate components, each belt member including a first end and a second and opposite end, the second end of each belt member being fixedly attached to the back portion of the chassis.

23. An absorbent garment as defined in claim 22, wherein the first end of each belt member includes a fastener that releasably attaches to the front portion of the chassis.

24. An absorbent garment as defined in claim 18, wherein the chassis includes a front edge and a back edge and the crotch portion defines a midline and wherein the distance from the midline to the front edge is at least about 5% less than the distance from the midline to the back edge.

25. An absorbent garment as defined in claim 18, wherein the chassis includes a front edge and a back edge and the crotch portion defines a midline and wherein the distance from the midline to the front edge is at least about 10% less than the distance from the midline to the back edge.
